(19) United States
(12) Patent Application Publication
(10) Pub. No.: US 2019/0292589 A1
(43) Pub. Date: Sep. 26, 2019

(54) Method and Apparatus for Analyzing Biomolecules

(71) Applicant: Hitachi High-Technologies Corporation, Minato-ku, Tokyo (JP)

(72) Inventors: Michiru FUJIOKA, Tokyo (JP); Yusuke GOTO, Tokyo (JP); Takahide YOKOI, Tokyo (JP)

(21) Appl. No.: 16/307,636

(22) PCT Filed: Jun. 10, 2016
(86) PCT No.: PCT/JP2016/067406

§ 371 Date: Dec. 6, 2018

(51) Int. Cl. C12Q 1/6869; G01N 27/447; G01N 33/487

(52) U.S. Cl. CPC C12Q 1/6869; G01N 33/48721; G01N 27/44791

(57) ABSTRACT

The purpose of the invention is to provide a method for analyzing biomolecules with which it is possible to easily suppress the occlusion of nanopores. The first embodiment of the invention is a method for analyzing biomolecules including a step for preparing a substrate having nanopores, a step for placing a sample solution including biomolecules and at least one compound selected from the group consisting of primary amines, secondary amines, guanidine compounds, and salts thereof on the substrate, and a step for detecting the changes in light or electrical signal generated when the biomolecules pass through the nanopores.

### TECHNICAL FIELD

The present invention relates to a method and an apparatus for analyzing biomolecules.

### BACKGROUND ART

Techniques for analyzing the base sequence of nucleic acids—a type of biomolecule—are very important for various purposes, including detection of causative genes of hereditary disease, evaluation of efficacy and side effects of drugs, and detection of genetic mutations associated with cancerous disease. Various apparatuses are available for the base sequence analysis of nucleic acids, including, for example, a fluorescence detection apparatus based on electrophoresis using capillary (3500 Genetic Analyzer; Thermo Fisher Scientific Inc.), and an apparatus that detects the fluorescence from nucleic acids immobilized on a plate (HiSeq 2500; illumina). However, these apparatuses require expensive fluorescence detectors and fluorescence reagents, and involve high costs.

In order to provide an analysis technique that can achieve detection at low cost, there is a study of a method that analyzes abase sequence by detecting changes in the light or electrical signal generated when a nucleic acid passes through a nanopore. For example, a hole (nanopore) of several nanometer size is formed through a 1 to 60 nm-thick thin membrane, using a transmission electron microscope. Tanks filled with an electrolyte solution are disposed on the both sides of the thin membrane, and electrodes are provided for these tanks. Applying a voltage across the electrodes passes an ion current through the nanopore. The ion current is roughly proportional to the cross sectional area of the nanopore. DNA blocks the nanopore as it passes through it, and makes the effective cross sectional area of the nanopore smaller, with the result that the ion current decreases. The term “block current” is used to describe such a change occurring in the ion current as a result of passage of DNA. The magnitude of block current can be used to distinguish between a single-stranded DNA and a double-stranded DNA, and the type of base. The subject of analysis by the technique using nanopores is not limited to DNA, and the technique can be used for the analysis of a range of biomolecules, for example, such as RNAs, peptides, and proteins. Because DNA is negatively charged, the DNA molecule passes through the nanopore from the negative electrode side to the positive electrode side.

Common knowledge is that a biological sample DNA contains adenine and guanine, which are bases with the purine skeleton, and cytosine and thymine (uracil in the case of RNA), which are bases with the pyrimidine skeleton. Adenine forms a hydrogen bond with thymine, and cytosine forms a hydrogen bond with guanine, and the hydrogen bonding between these bases forms the double helix structure of DNA. The hydrogen bonding is also responsible for the self-ligation of a single-stranded DNA forming a higher-order structure. The DNA double helix structure, and a higher-order structure of a single-stranded DNA create large steric hinderance in their passage through nanopores, and clogging may occur in the nanopores because of these DNA structures.

Concerning the issue of clogging, PTL 1 describes a technique intended to overcome the clogging of nanopores by irradiating nanopores with a laser provided as a heat source.

### CITATION LIST

### Patent Literature

PTL 1: US Patent Application 2013/0220811

### SUMMARY OF INVENTION

### Technical Problem

As described above, the technique that analyzes biomolecules such as nucleic acids with the use of nanopores involves clogging of nanopores with biomolecules. As a countermeasure, PTL 1 proposes a technique for solving this problem by means of laser irradiation. However, installing a device for laser irradiation adds to the cost and the structural complexity of the analysis apparatus. The generated heat of laser irradiation also may cause fast Brownian motion in the biomolecules. Biomolecules with fast Brownian motion move more rapidly in passing through a nanopore. This makes the block current value unstable, and accurate detection of the biological component becomes difficult. There accordingly is a need for a novel technique that can conveniently reduce clogging of nanopores without using a specialized mechanism such as a laser irradiator.

An object of the present invention is to provide a biomolecule analysis method capable of conveniently reducing clogging of nanopores.

### Solution to Problem

(1) A method for analyzing a biomolecule,

the method comprising the steps of:

preparing a substrate having a nanopore;

placing on the substrate a sample solution containing a biomolecule, and at least one compound (A) selected from the group consisting of primary amines, secondary amines, guanidine compounds, and salts thereof; and detecting a change in a light or electrical signal that occurs when the biomolecule passes through the nanopore.

(2) The method for analyzing a biomolecule according to (1), wherein the compound (A) is a primary amine represented by the following formula (I), or a salt thereof:

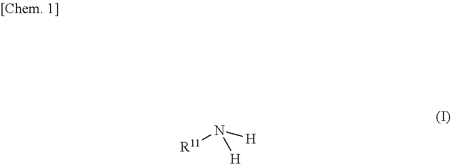

In the formula, R¹¹ is a substituted or unsubstituted alkyl group of 1 to 6 carbon atoms.

(3) The method for analyzing a biomolecule according to (1), wherein the compound (A) is a secondary amine represented by the following formula (II), or a salt thereof:

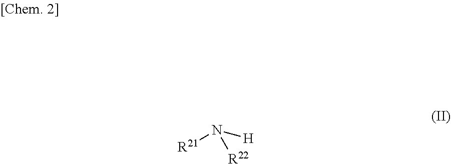

In the formula, R²¹ and R²² are each independently a substituted or unsubstituted alkyl group of 1 to 6 carbon atoms.

(4) The method for analyzing a biomolecule according to (1), wherein the compound (A) is a guanidine compound represented by the following formula (III), or a salt thereof :

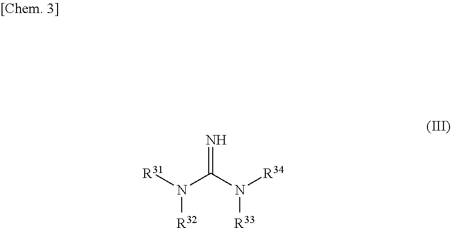

In the formula, R³¹, R³², R³³, and R³⁴ are each independently a hydrogen atom, a substituted or unsubstituted alkyl group of 1 to 6 carbon atoms, a cyano group, or an amino group.

(5) The method for analyzing a biomolecule according to (1), wherein the compound (A) is a monomethylamine or a salt thereof, a monoethylamine or a salt thereof, a dimethylamine or a salt thereof, or a diethylamine or a salt thereof.

(6) The method for analyzing a biomolecule according to (1), wherein the compound (A) guanidine or a salt thereof, monoaminoguanidine or a salt thereof, or diaminoguanidine or a salt thereof.

(7) The method for analyzing a biomolecule according to any one of (1) to (6), wherein the sample solution has a pH of 7.2 or more.

(8) The method for analyzing a biomolecule according to any one of (1) to (6), wherein the sample solution has a pH of 8.4 or more.

(9) A method for analyzing a biomolecule,

the method comprising the steps of:

preparing a substrate having a nanopore;

contacting the substrate to a solution containing at least one compound (A) selected from the group consisting of primary amines, secondary amines, guanidine compounds, and salts thereof;

placing a biomolecule-containing sample solution on the substrate brought into contact with the solution; and

detecting a chance in a light or electrical signal that occurs when the biomolecule passes through the nanopore.

(10) The method for analyzing a biomolecule according to (9), wherein the compound (A) is a primary amine represented by the following formula (I), or a salt thereof:

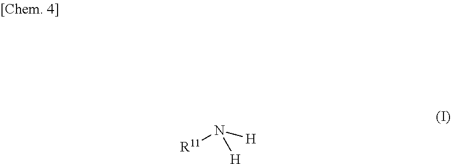

In the formula, R¹¹ is a substituted or unsubstituted alkyl group of 1 to 6 carbon atoms.

(11) The method for analyzing a biomolecule according to (9), wherein the compound (A) is a secondary amine represented by the following formula (II), or a salt thereof:

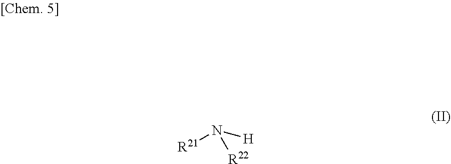

In the formula, R²¹ and R²² are each independently a substituted or unsubstituted alkyl group of 1 to 6 carbon atoms .

(12) The method for analyzing a biomolecule according to (9), wherein the compound (A) is a guanidine compound represented by the following formula (III), or a salt thereof:

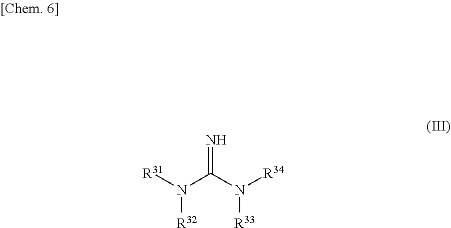

In the formula, R³¹, R³², R³³, and R³⁴ are each independently a hydrogen atom, a substituted or unsubstituted alkyl group of 1 to 6 carbon atoms, a cyano group, or an amino group.

(13) An apparatus for analyzing a biomolecule,

the apparatus comprising:

a sample introducing region;

a sample outflow region which a biomolecule flows into from the sample introducing region;

a substrate disposed between the sample introducing region and the sample outflow region and having a nanopore through which the biomolecule passes from the sample introducing region to the sample outflow region; and

a detecting section that detects a change in a light or electrical signal that occurs when the biomolecule passes through the nanopore,

the sample introducing region retaining a sample solution that contains the biomolecule, and at least one compound (A) selected from the group consisting of primary amines, secondary amines, guanidine compounds, and salts thereof.

(14) A solution for use in a method that analyzes a biomolecule by detecting a change in a light or electrical signal that occurs when the biomolecule passes through a nanopore,

the solution comprising at least one compound (A) selected from the group consisting of primary amines, secondary amines, guanidine compounds, and salts thereof.

### Advantageous Effects of Invention

The present invention can provide a biomolecule analysis method capable of conveniently reducing clogging of nanopores.

### BRIEF DESCRIPTION OF DRAWINGS

FIG. 1 is a schematic cross sectional view describing a configuration of a chamber section of a nanopore-type analysis apparatus equipped with a substrate having a nanopore.

### DESCRIPTION OF EMBODIMENTS

As used herein, the term “biomolecules” refers to biopolymers present in an organism, for example, such as nucleic acids (e.g., DNA, RNA), peptides, polypeptides, proteins, and sugar chains. The nucleic acids include single-stranded, double-stranded, and triple-stranded DNAs and RNAs, and any chemically modified form thereof.

As used herein, the term “analysis” means determination of characteristics of a biomolecule, or detection or identification of a biomolecule. For example, “analysis” means determination of the sequence of the constituents of a biomolecule. As used herein, “sequencing of a biomolecule” refers to determining the sequence or order of the constituents (bases) of a biomolecule (for example, DNA or RNA).

As used herein, the term “nanopore” refers to a hole of a nano-order size (specifically, a diameter of 0.5 nm or more and less than 1 μl). The nanopore is provided through a substrate, and is in communication with a sample introducing region and a sample outflow region.

The present invention is concerned with a method for analyzing a biomolecule that detects a change in the light or electrical signal that occurs when the biomolecule passes through a nanopore, using a substrate having a nanopore (hereinafter, also referred to as “nanopore substrate”). More specifically, the present invention is concerned with a method for determining the base sequence of a nucleic acid with a nucleic acid sequencer (also referred to as “nanopore sequencer”) having a nanopore substrate.

Embodiments of the present invention are described below in detail, with reference to the accompanying drawing.

### First Embodiment

First Embodiment of the present invention is a method for analyzing a biomolecule,

the method comprising the steps of:

preparing a substrate having a nanopore;

placing on the substrate a sample solution containing a biomolecule, and at least one compound (A) selected from the group consisting of primary amines, secondary amines, guanidine compounds, and salts thereof; and

detecting a change in a light or electrical signal that occurs when the biomolecule passes through the nanopore.

In the present embodiment, a biomolecule is analyzed by using a sample solution that contains a sample biomolecule, and at least one compound (A) selected from the group consisting of primary amines, secondary amines, guanidine compounds, and salts thereof. Clogging of nanopores can be reduced with the use of the sample solution containing compound (A).

### Sample Solution

Compound (A) is at least one selected from the group consisting of primary amines, secondary amines, guanidine compounds, and salts thereof. Clogging of nanopores can be reduced when the sample solution used for measurement contains compound (A).

The primary amines are compounds with one of the hydrogen atoms of ammonia substituted with a hydrocarbon group (preferably, an alkyl group). The hydrocarbon group has preferably 1 to 6 carbon atoms, preferably 1 to 4 carbon atoms, more preferably 1 to 3 carbon atoms. The hydrocarbon group may have a substituent. Examples of the substituent include an amino group (—NH₂). Preferably, the primary amines do not have the guanidine skeleton.

Preferably, the primary amines are compounds represented by the following formula (I).

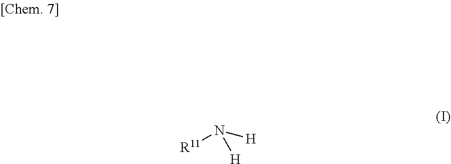

In formula (I), R¹¹ is a substituted or unsubstituted alkyl group of 1 to 6 carbon atoms.

The secondary amines are compounds with two of the hydrogen atoms of ammonia substituted with a hydrocarbon group (preferably, an alkyl group). The hydrocarbon group has preferably 1 to 6 carbon atoms, preferably 1 to 4 carbon atoms, more preferably 1 to 3 carbon atoms. The hydrocarbon group may have a substituent. Examples of the substituent include an amino group (—NH₂). Preferably, the secondary amines do not have the guanidine skeleton.

Preferably, the secondary amines are compounds represented by the following formula (II).

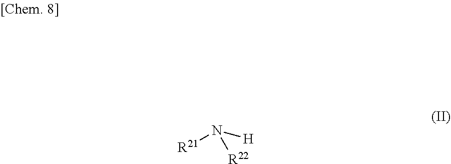

In formula (II), R²¹ and R²² are each independently a substituted or unsubstituted alkyl group of 1 to 6 carbon atoms.

The guanidine compounds are compounds having the guanidine skeleton HN═C (NR′R″)₂. R′ and R″ are independent from each other, and may be, for example, a hydrogen atom, a hydrocarbon group (preferably, an alkyl group), an amino group, or a cyano group. The hydrocarbon group has preferably 1 to 6 carbon atoms, preferably 1 to 4 carbon atoms, more preferably 1 to 3 carbon atoms. The hydrocarbon group may have a substituent. Examples of the substituent include an amino group (—NH₂).

Preferably, the guanidine compounds are compounds represented by the following formula (III).

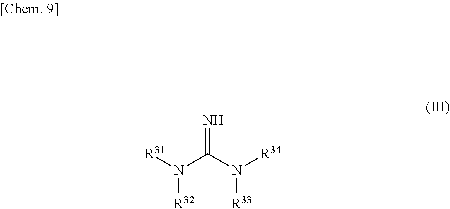

In the formula, R³¹, R³², R³³, and R³⁴ are each independently a hydrogen atom, a substituted or unsubstituted alkyl group of 1 to 6 carbon atoms, a cyano group, or an amino group.

In formulae (I) to (III), the alkyl group may be linear or branched, or may be cyclic. Preferably, the alkyl group is linear or branched. The alkyl group has preferably 1 to 4 carbon atoms, more preferably 1 to 3 carbon atoms, further preferably 1 to 2 carbon atoms. The alkyl group is preferably methyl or ethyl, more preferably methyl.

In formulae (I) to (III), the substituent of the alkyl group is preferably an amino group (—NH₂).

Preferred examples of the primary amines include monomethylamine and monoethylamine. Preferred examples of the secondary amines include dimethylamine and diethylamine. Preferred examples of the guanidine compounds include guanidine, monoaminoguanidine, and diaminoguanidine. That is, preferred examples of the compound (A) include monomethylamine or a salt thereof, monoethylamine or a salt thereof, dimethylamine or a salt thereof, diethylamine or a salt thereof, guanidine or a salt thereof, monoaminoguanidine or a salt thereof, and diaminoguanidine or a salt thereof.

Examples of the salts of primary amines, secondary amines, or guanidine compounds include hydrochlorides, thiocyanates, sulfates, phosphates, nitrates, and carbonates.

The compound (A) may be used alone, or in a combination of two or more.

The concentration of compound (A) in a sample solution is not particularly limited, and is, for example, 0.1 to 10 M, preferably 1 to 8 M, more preferably 2 to 6 M.

The sample solution has a pH of preferably 7.5 or more, more preferably 8.0 or more, further preferably 8.4 or more. With a pH of 7.5 or more, or a pH of 8.0 or more, clogging of nanopores can be more effectively reduced. The sample solution has a pH of preferably 11.0 or less, more preferably 10.0 or less. With a pH of 11.0 or less, damage to the nanopore substrate can be reduced.

The sample solution may contain a solvent such as water, and additives, in addition to a sample biomolecule, and compound (A). Examples of the additives include a buffer, and an electrolyte. The buffer may be appropriately selected according to the properties of the biomolecule. Examples of the buffer include Tris, trishydrochloride (Tris-HCl), and phosphate buffers. Particularly preferred are Tris and trishydrochloride because these buffers allow the sample solution to be controlled in a pH range of 7.5 or more with ease. The electrolyte (excluding the compound (A)) is a compound capable of generating ion current. For example, the electrolyte is potassium chloride or sodium chloride. The electrolyte concentration is, for example, 0.1 to 3 M.

### Analysis Apparatus

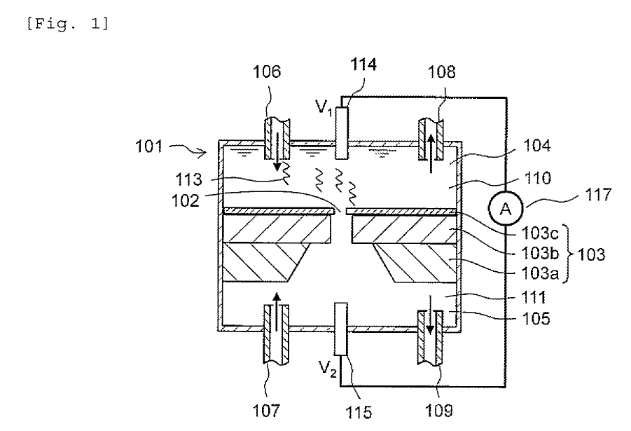

FIG. 1 is a schematic cross sectional view describing an exemplary structure of a chamber section of a nanopore-type analysis apparatus that can be used for the analysis method according to the present invention. In FIG. 1, a chamber section 101 includes a sample introducing region 104, a sample outflow region 105, and a substrate (nanopore substrate) 103 disposed between the sample introducing region 104 and the sample outflow region 105 and having a nanopore 102. The sample introducing region 104 and the sample outflow region 105 are spatially in communication with each other via the nanopore 102, allowing a biomolecule (sample 113) to move from the sample introducing region 104 to the sample outflow region 105 through the nanopore 102. A first liquid 110 fills the sample introducing region 104 via a first inflow channel 106. A second liquid 111 fills the sample outflow region 105 via a second inflow channel 107. The first liquid 110 and the second liquid 111 may flow out of the sample introducing region 104 and the sample outflow region 105, respectively, via a first outflow channel 108 and a second outflow channel 109. During analysis, the first liquid 110 and the second liquid 111 may or may not flow from the inflow channels to the outflow channels. The first inflow channel 106 and the second inflow channel 107 may be provided in positions opposite each other via the substrate. Similarly, the first outflow channel 108 and the second outflow channel 109 may be provided in positions opposite each other via the substrate.

In an embodiment, the substrate 103 includes a base (base material) 103a, and a thin membrane 103b formed on the base 103a. The substrate 103 may include an insulating layer 103c formed on the thin membrane 103b. The nanopore is formed in the thin membrane 103b. The nanopore can be easily and efficiently provided for the substrate by forming the thin membrane on the base 103a with a material and in a thickness suited for the formation of nanopore. Examples of the thin membrane material include graphene, silicon, silicon compounds (for example, silicon oxide, silicon nitride, silicon oxynitride), metal oxides, and metal silicates. In a preferred embodiment, the thin membrane is formed of a material containing silicon or a silicon compound. The thin membrane (or, in some cases, the whole substrate) may be substantially transparent. Here, “substantially transparent” means that the membrane passes at least about 50%, preferably at least 80% of external light. The thin membrane may be a monolayer or a multilayer. The thin membrane has a thickness of 0.1 nm to 200 nm, preferably 0.1 nm to 50 nm, more preferably 0.1 nm to 20 nm. The thin membrane can be formed by using techniques known in the art, for example, such as low pressure chemical vapor deposition (LPCVD).

In the present invention, the sample solution is at least the first liquid 110. That is, the first liquid 110 is a sample solution containing biomolecules (sample 113) and compound (A). The second liquid 111 may also contain biomolecules and compound (A). In the present embodiment, the first liquid 110 may contain a solvent (preferably, water), and an electrolyte (for example, KCl or NaCl), in addition to biomolecules and compound (A). Ions originating in the electrolyte can serve to provide charge. The electrolyte is preferably a substance with a high degree of ionization, and may be, for example, potassium chloride or sodium chloride, as mentioned above.

The chamber section 101 is provided with a first electrode 114 and a second electrode 115, which are disposed in the sample introducing region 104 and the sample outflow region 105, respectively, opposite each other via the nanopore 102. In the present embodiment, the chamber section also includes a voltage applying section for the first electrode 114 and the second electrode 115. In response to an applied voltage, the charged sample 113 moves from the sample introducing region 104 to the sample outflow region 105 through the nanopore 102.

In addition to the chamber section, the nanopore-type analysis apparatus may include a detecting section that detects a change in the light or electrical signal that occurs when the biomolecule passes through the nanopore. The detecting section may include an amplifier for amplifying an electrical signal, an analog-digital converter for converting the analog output of the amplifier into a digital output, and a recorder for recording the measured data.

The method of detecting a change in the light or electrical signal that occurs when the biomolecule passes through the nanopore is not particularly limited, and, for example, a known detection method may be used. Specific examples of such detection methods include a block current method, a tunnel current method, and a capacitance method. As an example, the following briefly describes a detection method using block current. A biomolecule (for example, a nucleic acid) blocks the nanopore as it passes through the nanopore. This limits the ion flow through the nanopore, and the current decreases (block current). The magnitude of the block current, and the duration of the block current can be measured to analyze the length and the base sequence of individual nucleic acid molecules passing through the nanopore. In the case of, for example, the tunnel current method, a biomolecule passing between the electrodes disposed near the nanopore can be detected in the form of a tunnel current.

An example of a method that detects light is a method that uses Raman light. For example, external light (excitation light) is applied to excite the biopolymer that has entered the nanopore, and generate Raman scattered light. The characteristics of the biopolymer can then be determined from the spectrum of the Raman scattered light. The measurement section may include a light source that applies external light, and a detector that detects the Raman scattered light (e.g., spectroscopic detector). A conductive thin membrane may be disposed near the nanopore, and a near field may be generated to enhance the light. The accuracy of analysis can improve when detection using a block current method, a tunnel current method, or a capacitance method is combined with detection using Raman light.

The substrate 103 has at least one nanopore. The substrate 103 may be formed of an electrically insulating material, which may be, for example, an inorganic material or an organic material (including polymer materials). Examples of the electrically insulating material of the substrate include silicon, silicon compounds, glass, quartz, polydimethylsiloxane (PDMS), polytetrafluoroethylene (PTFE), polystyrene, and polypropylene. Examples of the silicon compounds include silicon nitride, silicon oxide, silicon carbide, and silicon oxynitride. The base (base material) as a support for the substrate may be formed by using any of these materials, preferably, for example, a material (silicon material) containing silicon or a silicon compound. Examples of the material of the nanopore-forming thin membrane include graphene, silicon, silicon compounds (for example, silicon oxide, silicon nitride, and silicon oxynitride), metal oxides, and metal silicates, as mentioned above. Preferred are materials containing silicon or a silicon compound. That is, in the present embodiment, it is preferable that the nanopore be formed in a member formed of a silicon- or silicon compound-containing material. The silicon- or silicon compound-containing material has a silanol group on its surface. In the method of the present invention, the compound (A) presumably acts on the silanol group, preventing the silanol group from being acted upon by nucleic acid. It is to be noted, however, that this presumption is not intended to limit the present invention

Preferably, the insulating layer 103c is provided on the thin membrane 103b. The insulating layer has a thickness of preferably 5 nm to 50 nm. The material of the insulating layer may be any insulating material, preferably a material containing, for example, silicon or a silicon compound (for example, silicon oxide, silicon nitride, and silicon oxynitride).

The substrate may be produced by using a method known in the art. Alternatively, the substrate may be a commercially available product. The substrate may be formed by using a technique, for example, such as photolithography, electron beam lithography, etching, laser ablation, injection molding, casting, a molecular beam epitaxy method, chemical vapor deposition (CVD), dielectric breakdown, and an electron beam or focused ion beam method.

The nanopore size may be appropriately selected according to the type of the biopolymer to be analyzed. The nanopores may have a uniform diameter, or the diameter may be different in different parts of the membrane. The nanopore may be connected to a pore having a diameter of 1 μm or more. The nanopore has a diameter of preferably 100 nm or less, preferably 1 nm to 100 nm, preferably 1 nm to 50 nm, preferably 1 nm to 10 nm.

Examples of the biomolecules to be analyzed include ssDNA (single-stranded DNA). The ssDNA has a diameter of about 1.5 nm, and the appropriate range of nanopore diameter for the analysis of ssDNA is 1.5 nm to 10 nm, preferably 1.5 nm to 2.5 nm. The dsDNA (double-stranded DNA) has a diameter of about 2.6 nm, and the appropriate range of nanopore diameter for the analysis of dsDNA is 3 nm to 10 nm, preferably 3 nm to 5 nm. For analysis of other biomolecules, for example, such as proteins, polypeptides, and sugar chains, the nanopore diameter also can be selected taking into consideration the dimensions of the biomolecules.

The nanopore depth (length) may be adjusted by adjusting the thickness of the nanopore-forming member (for example, the thickness of the thin membrane 103b). Preferably, the nanopore has the same depth as the monomer unit of the biomolecule to be analyzed. For example, when the biomolecule is a nucleic acid, the nanopore has a depth no greater than a single base, for example, about 0.3 nm or less. The nanopore is basically circular in shape. However, the nanopore may have an elliptical or polygonal shape.

The substrate may have at least one nanopore. When more than one nanopore is provided, the nanopores may be orderly arranged. The nanopore may be formed by using a method known in the art, for example, a method that applies an electron beam from a transmission electron microscope (TEM), or a nanolithography technique or an ion beam lithography technique. The nanopore also may be formed in the substrate by using electrical breakdown.

As described above, the chamber section 101 may include the first electrode 114 and the second electrode 115 that cause passage of the sample 113 through the nanopore 102, in addition to the sample introducing region 104, the sample outflow region 105, and the substrate 103. In a preferred example, the chamber section 101 includes the first electrode 114 provided in the sample introducing region 104, the second electrode 115 provided in the sample outflow region 105, and the voltage applying section that applies voltage to the first electrode and the second electrode. An ammeter 117 may be disposed between the first electrode 114 provided in the sample introducing region 104, and the second electrode 115 provided in the sample outflow region 105. The passage speed of the sample through the nanopore can be adjusted with the current between the first electrode 114 and the second electrode 115. The current value can be appropriately selected by a skilled person. When the sample is a DNA, the current value is preferably 100 mV to 300 mV.

The electrode material may be metal. Examples of the metal include the platinum-group metals such as platinum, palladium, rhodium, and ruthenium; gold, silver, copper, aluminum, nickel; and graphite (may be a monolayer or a multilayer), for example, such as graphene; tungsten, and tantalum.

### Second Embodiment

Second Embodiment of the present invention is a biomolecule analysis method that includes the steps of:

preparing a substrate having a nanopore;

contacting the substrate to a solution containing at least one compound (A) selected from the group consisting of primary amines, secondary amines, guanidine compounds, and salts thereof;

placing a biomolecule-containing sample solution on the substrate brought into contact with the solution; and

detecting a change in a light or electrical signal that occurs when the biomolecule passes through the nanopore.

In the present embodiment, clogging of the nanopore can be reduced by detecting a sample with the nanopore substrate brought into contact with the solution containing compound (A), preferably the nanopore substrate dipped in the solution containing compound (A). The reduced clogging of nanopore is probably due to some desirable effect on sample measurement as a result of the compound (A) adhering to the wall surface of the nanopore after the adhesion of the compound (A) to the wall surface of the nanopore or to substrate surfaces around nanopores upon the nanopore substrate contacting the solution containing compound (A). However, this presumption is not intended to limit the present invention.

### Third Embodiment

Third Embodiment of the present invention is a biomolecule analysis apparatus that includes:

a sample introducing region;

a sample outflow region which the biomolecule flows into from the sample introducing region;

a substrate disposed between the sample introducing region and the sample outflow region and having a nanopore through which the biomolecule passes from the sample introducing region to the sample outflow region; and

a detecting section that detects a change in a light or electrical signal that occurs when the biomolecule passes through the nanopore,

the sample introducing region retaining a sample solution that contains the biomolecule, and at least one compound (A) selected from the group consisting of primary amines, secondary amines, guanidine compounds, and salts thereof.

### Fourth Embodiment

Fourth Embodiment of the present invention is a solution for use in a method that analyzes a biomolecule by detecting a change in a light or electrical signal that occurs when the biomolecule passes through a nanopore, and the solution includes at least one compound (A) selected from the group consisting of primary amines, secondary amines, guanidine compounds, and salts thereof.

The solution according to the present embodiment can be used for the analysis method of First Embodiment after being prepared as a sample solution by adding a component (e.g., a sample). The solution according to the present embodiment can be used for the analysis method of Second Embodiment by dipping the nanopore substrate in the solution.

### EXAMPLES

The present invention is described below in greater detail by way of Examples. It is to be noted, however, that the present invention is in no way limited by the following Examples.

### Example A

Example A describes an example of First Embodiment of the present invention.

### Sample

A several kilo to several tens of kilobase-long DNA was prepared as a sample, using the following method. First, a sequence A₅₀T₂₅C₂₅ (single-stranded DNA) was synthesized that had 50 contiguous adenine residues, 25 contiguous thymine residues, and 25 contiguous cytosine residues. The synthesized single-stranded DNA was transformed into a cyclic form using a single-stranded DNA ligase (CircLigase® ssDNA Ligase; ARBROWN Co., Ltd.), and amplified into a long-chain (several kilo to several tens of kilobases) DNA, using phi29 DNA Polymerase (New England BioLabs). Because of the contiguous adenine and thymine sequence, the synthesized DNA can form a higher-order structure through self hybridization with relative ease. This makes the DNA desirable for evaluation in the present invention.

### Sample Solution

In Example A, eight sample solutions (aqueous solutions) were prepared, as follows. The sample solutions each contained the sample single-stranded DNA in a concentration of 1 ng/μl.
Sample solution E1: 2 M 1,3-diaminoguanidine hydrochloride, 0.1 M Tris
Sample solution E2: 6 M guanidine hydrochloride, 0.1 M Tris
Sample solution E3: 4 M diethylaminehydrochloride, 0. 1 M Tris
Sample solution E4: 6 M methylamine hydrochloride, 0.1M Tris
Sample solution E5: 4 M dimethylamine hydrochloride, 0.1 N Tris
Sample solution C1: 1 M potassium chloride, 10 mM Tris-HCl, 1 mM EDTA
Sample solution C2: 1 M potassium chloride, 0.1 M Tris
Sample solution C3: 4 M trimethylamine hydrochloride, 0.1 M Tris
Tris (trishydroxymethylaminomethane)

The sample solutions C1 and C2 had compositions commonly used for nanopore-type DNA sequences.

### Example A1

Sample solution E1 was placed in the sample introducing region 104 of the nanopore-type analysis apparatus of the configuration shown in FIG. 1, and the block current generated by the passage through the nanopore 102 was measured. The nanopore size was 1.4 to 2.0 nm. A patch clamp amplifier (Axopatch 200B amplifiers; Molecular Devices) was used for the detection of block current. The block current was detected at a sampling rate of 50 kHz under an applied voltage of +300 mV. The result was evaluated from the detected data with regard to “clogging”, “number of events “, “number of prolonged blockage”, and “frequency”. Here, the number of events indicates the number of passages of the single-stranded DNA through the nanopore. The number of prolonged blockage indicates the number of times the current remained at a reduced value for at least 5 seconds. The frequency was calculated using the formula: Number of Prolonged Blockage/Number of Events×100 (%). When the current remained at a reduced value for 5 seconds or more, the voltage was inverted to −300 mV to clear the DNA blocking the nanopore. Clogging was determined as being present when the nanopore remained blocked even after the voltage was inverted.

### Example A2

The block current was measured, and evaluation was made in the same manner as in Example A1, except that sample solution E2 was used instead of sample solution E1.

### Example A3

The block current was measured, and evaluation was made in the same manner as in Example A1, except that sample solution E3 was used instead of sample solution E1.

### Example A4

The block current was measured, and evaluation was made in the same manner as in Example A1, except that sample solution E4 was used instead of sample solution E1.

### Example A5

The block current was measured, and evaluation was made in the same manner as in Example A1, except that sample solution E5 was used instead of sample solution E1.

### Comparative Example A1

The block current was measured, and evaluation was made in the same manner as in Example A1, except that sample solution C1 was used instead of sample solution E1.

### Comparative Example A2

The block current was measured, and evaluation was made in the same manner as in Example A1, except that sample solution C2 was used instead of sample solution E1.

### Comparative Example A3

The block current was measured, and evaluation was made in the same manner as in Example A1, except that sample solution C3 was used instead of sample solution E1.

The results of the evaluations for Examples A1 to A5 and Comparative Examples A1 to A3 are presented in Table 1.

TABLE 1

| | | | | | | Number | |
|---|---|---|---|---|---|---|---|
| | | | Electrical | | Number | of | |
| | Sample | Solution | conductivity | | of | prolonged | Frequency |
| | solution | pH | [ms/cm] | Clogging | events | blockage | [%] |
| Example A1 | E1 | 9.2 | 124.9 | Absent | 261 | 8 | 3.1 |
| Example A2 | E2 | 9.6 | 242.0 | Absent | 1350 | 38 | 2.8 |
| Example A3 | E3 | 8.7 | 82.0 | Absent | 482 | 13 | 2.7 |
| Example A4 | E4 | 8.4 | 302.4 | Absent | 1083 | 4 | 0.37 |
| Example A5 | E5 | 8.7 | 214.5 | Absent | 1493 | 4 | 0.27 |
| Comparative | C1 | 7.5 | — | Present | — | — | — |
| Example A1 | | | | | | | |
| Comparative | C2 | 10.3 | 111.2 | Present | — | — | — |
| Example A2 | | | | | | | |
| Comparative | C3 | 8.2 | 161.3 | Present | — | — | — |
| Example A3 | | | | | | | |

In Comparative Examples A1to A3, clogging occurred in the nanopores, though some DNA passage events took place. Clogging of nanopores did not occur in Examples A1 to A5.

### Example B

Example B describes an example of Second Embodiment of the present invention.

### Example B1

Solution E6 (an aqueous solution containing 4 M dimethylamine hydrochloride and 0.1 M Tris) was added to the sample introducing region 104 of the nanopore-type analysis apparatus of the configuration shown in FIG. 1, and the nanopore substrate was dipped in the solution E6 for 30 minutes. The solution E6 was then removed from the sample introducing region. A sample solution (hereinafter, “sample solution E7”) containing a PolyG sequence of 30 contiguous guanine residues that easily transforms into a higher-order structure was injected as a sample DNA into a 4 M dimethylamine hydrochloride and 0.1 M Tris solution in the sample introducing region 104. The block current was measured, and evaluation was made under the same conditions used for Example A1.

### Reference Example B1

The block current was measured, and evaluation was made in the same manner as in Example A1, except that sample solution E7 was injected into the sample introducing region 104 without dipping the nanopore substrate in solution E6.

### Example B2

The nanopore substrate was dipped in solution E6 in the same manner as in Example B1, and a sample solution (hereinafter, “sample solution E8”) containing a PolyG sequence of 30 contiguous guanine residues that easily transforms into a higher-order structure was injected as a sample DNA into a 6 M guanidine hydrochloride and 0.1 M Tris solution in the sample introducing region 104. The block current was measured, and evaluation was made under the same conditions used for Example A1.

### Reference Example B2

The block current was measured, and evaluation was made in the same manner as in Example A1, except that sample solution E8 was injected into the sample introducing region 104 without dipping the nanopore substrate in solution E6.

The results of the evaluations for Examples E1 and B2 and Reference Examples B1 and B2 are presented in Table 2.

TABLE 2

| | | | | | | Number | |
|---|---|---|---|---|---|---|---|
| | | pH of | | | Number | of | |
| | Dipping | dipping | Sample | | of | prolonged | Frequency |
| | solution | solution | solution | Clogging | events | blockage | [%] |
| Example B1 | E6 | 8.7 | E7 | Absent | 549 | 1 | 0.18 |
| Reference | — | — | E7 | Absent | 957 | 5 | 0.52 |
| Example B1 | | | | | | | |
| Example B2 | E6 | 8.7 | E8 | Absent | 2245 | 3 | 0.13 |
| Reference | — | — | E8 | Absent | 596 | 18 | 3.0 |
| Example B2 | | | | | | | |

By comparing Example B1 and Reference Example B1, and Example B2 and Reference Example B2, it can be seen that the number or frequency of prolonged blockage of the nanopore by the sample is smaller when the nanopore substrate, prior to the measurement, is dipped in the solution containing compound (A). As can be understood from this result, clogging of nanopore can be reduced also in Second Embodiment of the present invention.

### REFERENCE SIGNS LIST

101 Chamber section
102 Nanopore
103 Substrate
103a Base (base material)
103b Thin membrane
103c Insulating layer
104 Sample introducing region
105 Sample outflow region
106 First inflow channel
107 Second inflow channel
108 First outflow channel
109 Second outflow channel
110 First liquid
111 Second liquid
113 Sample (biomolecule)
114 First electrode
115 Second electrode
117 Power supply with ammeter

1.-14. (canceled)

15. A method for analyzing a nucleic acid,
the method comprising the steps of:
preparing a substrate having a nanopore;
placing on the substrate a sample solution containing a nucleic acid, and at least one compound (A) selected from the group consisting of primary amines, secondary amines, and salts thereof; and
detecting a change in light or an electrical signal that occurs when the nucleic acid passes through the nanopore.

16. The method for analyzing a nucleic acid according to claim 15, wherein the compound (A) is a primary amine represented by the following formula (I), or a salt thereof: 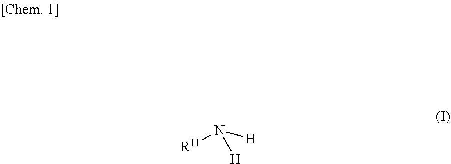
wherein R¹¹ is a substituted or unsubstituted alkyl group of 1 to 6 carbon atoms.

17. The method for analyzing a nucleic acid according to claim 15, wherein the compound (A) is a secondary amine represented by the following formula (II), or a salt thereof: 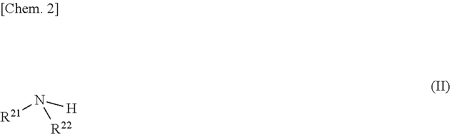
wherein R²¹ and R²² are each independently a substituted or unsubstituted alkyl group of 1 to 6 carbon atoms.

18. The method for analyzing a nucleic acid according to claim 15, wherein the compound (A) is a monomethylamine or a salt thereof, a monoethylamine or a salt thereof, a dimethylamine or a salt thereof, or a diethylamine or a salt thereof.

19. The method for analyzing a nucleic acid according to claim 15, wherein the sample solution has a pH of 7.2 or more.

20. The method for analyzing a nucleic acid according to claim 15, wherein the sample solution has a pH of 8.4 or more.

21. A method for analyzing a biomolecule,
the method comprising the steps of:
(1) preparing a substrate having a nanopore;
(2) contacting the substrate to a solution containing at least one compound (A) selected from the group consisting of primary amines, secondary amines, guanidine compounds, and salts thereof, and causing the compound (A) to adhere to the substrate;
(3) removing the solution from the substrate;
(4) placing a biomolecule-containing sample solution on the substrate having the compound (A) adhering to the substrate; and
(5) detecting a change in light or an electrical signal that occurs when the biomolecule passes through the nanopore,
the steps (1) to (5) being performed in this order.

22. The method for analyzing a biomolecule according to claim 21, wherein the compound (A) is a primary amine represented by the following formula (I), or a salt thereof: 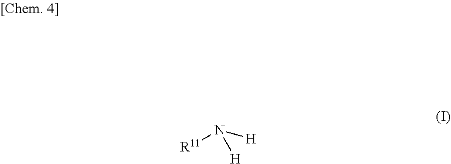
wherein R¹¹ is a substituted or unsubstituted alkyl group of 1 to 6 carbon atoms.

23. The method for analyzing a biomolecule according to claim 21, wherein the compound (A) is a secondary amine represented by the following formula (II), or a salt thereof: 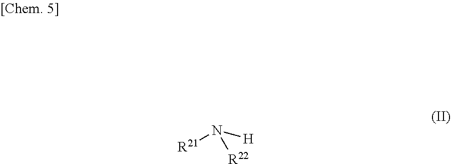
wherein R²¹ and R²² are each independently a substituted or unsubstituted alkyl group of 1 to 6 carbon atoms.

24. The method for analyzing a biomolecule according to claim 21, wherein the compound (A) is a guanidine compound represented by the following formula (III), or a salt thereof: 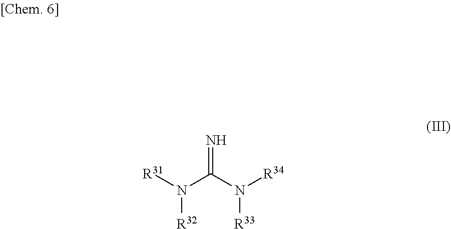
wherein R³¹, R³², R³³, and R³⁴ are each independently a hydrogen atom, a substituted or unsubstituted alkyl group of 1 to 6 carbon atoms, a cyano group, or an amino group.

25. An apparatus for analyzing a nucleic acid,
the apparatus comprising:
a sample introducing region;
a sample outflow region which a nucleic acid flows into from the sample introducing region;
a substrate disposed between the sample introducing region and the sample outflow region and having a nanopore through which the nucleic acid passes from the sample introducing region to the sample outflow region; and
a detecting section that detects a change in light or an electrical signal that occurs when the nucleic acid passes through the nanopore,
the sample introducing region retaining a sample solution that contains the nucleic acid, and at least one compound (A) selected from the group consisting of primary amines, secondary amines, and salts thereof.

26. A solution for use in a method that analyzes a nucleic acid by detecting a change in light or an electrical signal that occurs when the nucleic acid passes through a nanopore,
the solution comprising at least one compound (A) selected from the group consisting of primary amines, secondary amines, and salts thereof.

27. A method for analyzing a biomolecule,
the method comprising the steps of:
preparing a substrate having a nanopore;
placing on the substrate a sample solution containing a biomolecule, and at least one compound (A) selected from the group consisting of primary amines and salts thereof; and
detecting a change in light or an electrical signal that occurs when the biomolecule passes through the nanopore.

28. The method for analyzing a nucleic acid according to claim 15, wherein the compound (A) is monomethylamine or a salt thereof, or dimethylamine or a salt thereof.
